(19) United States
(12) Patent Application Publication
(10) Pub. No.: US 2023/0335247 A1
(43) Pub. Date: Oct. 19, 2023

(54) System and Method for the Portable Algorithmic Cultivation and Reformulation of Psilocybin

(71) Applicant: Nima Veiseh, Grand Haven, MI (US)

(72) Inventor: Nima Veiseh, Grand Haven, MI (US)

(21) Appl. No.: 18/302,454

(22) Filed: Apr. 18, 2023

Related U.S. Application Data
(60) Provisional application No. 63/332,203, filed on Apr. 18, 2022.

(51) Int. Cl. G16H 20/10

(52) U.S. Cl. CPC G16H 20/10

(57) ABSTRACT

Disclosed is a system and method of portable algorithmic cultivation and reformulation of psilocybin.

The current application claims a priority to the U.S. provisional patent application serial number 63/332,203 filed on Apr. 18, 2022.

### FIELD OF THE INVENTION

The present invention generally relates to a method for cultivation and utilizing blockchain enabled process. More specifically, the present invention is a method for a cultivation that uses a blockchain enabled process for real-time clinical feedback about mushrooms, reformulation in real-time safely quality in terms of quality assurance and therapeutic efficacy for the mushrooms.

### BACKGROUND OF THE INVENTION

The present invention, for the psilocybin and related active pharmaceutical ingredient space, provides a cultivation reformulation methodology and safety methodology including, but not limited to, a clinical setting, for the use of increasing quality and clinical efficacy of manufactured products. This works to ensure that psilocybin can be reformulated safely and administered to patients or taken recreationally.

### BRIEF DESCRIPTION OF THE DRAWINGS

FIG. 1 is a diagram of the present invention.

### DETAIL DESCRIPTIONS OF THE INVENTION

All illustrations of the drawings are for the purpose of describing selected versions of the present invention and are not intended to limit the scope of the present invention.

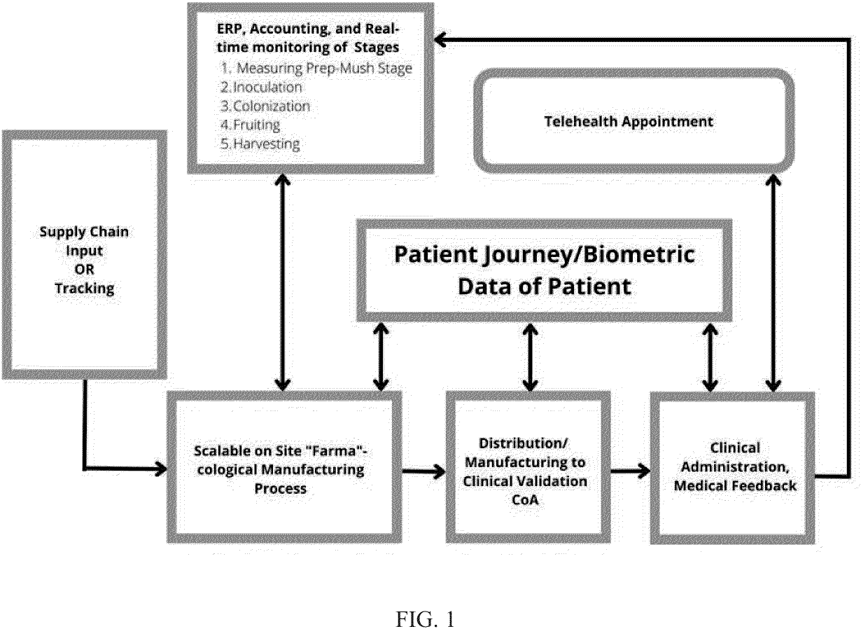

The present invention cultivation process for the growth of psilocybin (including but not limited to psychedelic, therapeutic) coupled with a blockchain-enabled process where real-time clinical feedback about mushrooms, reformulation, real-time safety, and quality assurance for the mushrooms. The cultivation methodology and structure is described in FIG. 1, and can be recursively or otherwise characterized, stored and/or transmitted digitally and securely. Clinical and therapeutic ethically monitoring is key, and the invention uses block-chain technology to preserve security and privacy of both parties, to help elevate the integrity of the data used and exchanged in order to maximize quality and impact of the resulting products.

Incorporating elements of blockchain for data integrity and to protect in a regulatory disjointed environment against holes in places like the MTRIC system (cannabis).

Clinical administration and teletherapy aspects of the invention include by are not limited to teleservices, IoT devices, desktop computers, wearables, etc., and the product not limited to psilocybin and psilocybin or related active pharmaceutical ingredients.

Each cycle produces a “pickled model” (or “spore model”) which is a serialized and portable representation of the farming instance, that can then be consolidated and transferred as its own algorithmic outcome or IP. Because portability and modularity are the cornerstones of scalability, the invention satisfies scalability enablement for cultivation both from personal and/or industrial cultivation scales. This spore can include, but is not limited to, industrial cultivation information, equipment settings, manufacturing inputs, manufacturing outputs, associated clinical outcomes, associated patients, genomic data the spores and/or psilocybin producing fungi. This helps to solve the problem of sustainable and scalable farming and production within an industry that is emerging from its nascency. This also helps to provide a decentralized market for the exchange of ideas and/or methods for the ongoing cultivation and improvement of the Active Pharmaceutical Ingredient or related byproducts. The Spore can also be used as a means for cultivators, investors and/or clinicians to validate elements of the cultivation process.

The present invention is the method of cultivation and the ability that emerges in the ability to scale, transfer, transmit, validate and/or store the qualities of the manufacturing process. The edges/arrows of the diagram (FIG. 1) have 3 distinct properties: 1) directional vector, 2) magnitude vector (of supply chain flow of data or physical assets), 3) regulatory buffer (is this possible from the existing regulatory standpoint). Seamless transfer, communication, and integrity of recipes stored on the blockchain, giving ownership and sovereignty to their originators.

The prior art for the present invention includes the following:
1. US10933073B2
2. NL2018190B1
3. US20150343144A1
4. US20180343812A1
5. US20220054402A1
6. EP3399972B1
7. US10327464B2
8. US8631126B2
9. WO2006091528A3
10. WO2009110991A2
11. US20220013202A1
12. “Markets With Memory”, by Nima Veiseh, 2019.ISBN: 9781700165121

Although the invention has been explained in relation to its preferred embodiment, it is to be understood that many other possible modifications and variations can be made without departing from the spirit and scope of the invention.

What is claimed is:

1. A method of portable algorithmic cultivation and reformulation of psilocybin, the method comprising the steps of:
(A) providing a wearable or interactive drug delivery and user journey logging device configured for wireless or wired communication with ecosystem connectivity;
(B) executing an algorithmic process for producing, reproducing, and subsequent reformulation of psilocybin, psilocin, baeocystin, norbaeocystin, salts thereof, combinations thereof, and/or related byproducts;
(C) identifying a reproducible and scalable means of stable farming methodologies that account for the disparate regulatory and/or market environments, both domestically and internationally, in the efforts to raise quality, quantity, efficacy, specificity, and other characteristics of the API’s outcomes;
(D) serializing, both openly or securely, the entirety of data and/or processes used to characterize the cultivation process as a digital spore;
(E) executing a gain of function of related active pharmaceutical or non-pharmacological components, compounds and sub compounds; and
(F) discerning an entourage effect and its changes over time, scoped at the patient, farm, systemic, or regulatory level.
